(19) United States
(12) Patent Application Publication
(10) Pub. No.: US 2014/0142185 A1
(43) Pub. Date: May 22, 2014

(54) 3-Benzyloxyphenyloxoacetic Acid Compounds for Reducing Uric Acid

(71) Applicants: Shalini Sharma, Gaithersburg, MD (US); Reid W. von Borstel, Potomac, MD (US)

(72) Inventors: Shalini Sharma, Gaithersburg, MD (US); Reid W. von Borstel, Potomac, MD (US)

(21) Appl. No.: 14/004,928

(22) PCT Filed: Mar. 12, 2012
(86) PCT No.: PCT/US12/28723

§ 371 Date: Jan. 17, 2014

(30) Foreign Application Priority Data
Mar. 14, 2011 (US) 61452410

(51) Int. Cl. C07C 59/215; A61K 45/06; A61K 31/192

(52) U.S. Cl. CPC C07C 59/215; A61K 31/192; A61K 45/06
USPC 514/570; 562/464

(57) ABSTRACT

Uric acid in mammalian subjects is reduced and excretion of uric acid is increased by administering a compound of Formula (I) or its pharmaceutically acceptable salts. The uric acid-lowering effects of the compounds of this invention are used to treat or prevent a variety of conditions including gout, hyperuricemia, elevated levels of uric acid that do not meet the levels customarily justifying a diagnosis of hyperuricemia, renal dysfunction, kidney stones, cardiovascular disease, risk for developing cardiovascular disease, tumor-lysis syndrome, cognitive impairment, early-onset essential hypertension, and Plasmodium falciparum-induced inflammation. R¹ is hydrogen or alkyl having from 1 to 3 carbon atoms. R² is alkyl having from 1 to 3 carbon atoms, alkoxy having from 1 to 3 carbon atoms, hydroxy, nitro, halo, thio, alkylthio, or cyano. R³ and R⁴ are each independently hydrogen, methyl, ethyl, perfluoromethyl, methoxy, ethoxy, perfluoromethoxy, halo, hydroxy, nitro, or amino.

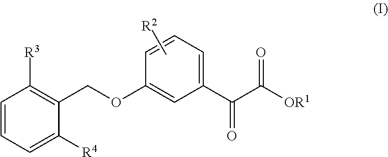

### BACKGROUND OF THE INVENTION

Diseases caused by elevated levels of uric acid fall into two major categories: disorders caused by precipitation of uric acid crystals and diseases related to pathological effects of soluble uric acid. Gouty arthritis is the classic example of the former. Deposition of urate crystals in the kidney is also a common cause of renal dysfunction. Elevated levels of soluble uric acid are associated with a variety of disorders, including cardiovascular and renal diseases.

There is a significant medical need for new medications that can safely, conveniently and effectively treat and prevent disorders related to elevation of blood uric acid, whether such diseases are due to crystallization of uric acid or to effects of supranormal (whether by an individual or a population-based standard) levels of soluble uric acid.

### SUMMARY OF THE INVENTION

This invention provides a compound represented by Formula I

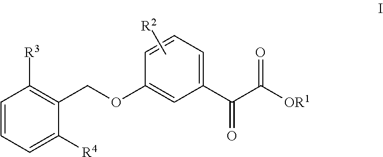

wherein R¹ is hydrogen or alkyl having from 1 to 3 carbon atoms; R² is alkyl having from 1 to 3 carbon atoms, alkoxy having from 1 to 3 carbon atoms, hydroxy, nitro, halo, thio, alkylthio, or cyano; R³ and R⁴ are each independently hydrogen, methyl, ethyl, perfluoromethyl, methoxy, ethoxy, perfluoromethoxy, halo, hydroxy, nitro, or amino; or a pharmaceutically acceptable salt of the compound.

This invention provides a method of reducing the uric acid concentration in blood of, or increasing uric acid excretion from, a mammalian subject, comprising administering to the subject a compound or salt of this invention in an amount effective to reduce the uric acid concentration in blood of, or increase uric acid excretion from, the subject. This invention provides a compound or salt of this invention for use in reducing the uric acid concentration in blood of, or increasing uric acid excretion from, a mammal. This invention provides the use of a compound or salt of this invention in the manufacture of a medicament for reducing the uric acid concentration in blood of, or increasing uric acid excretion from, a mammal. This invention provides a pharmaceutical composition for use in reducing the uric acid concentration in blood of, or increasing uric acid excretion from, a mammalian subject comprising a compound or salt of this invention in an amount effective to reduce the uric acid concentration in blood of, or increase uric acid excretion from, the subject. This invention provides a kit comprising one or more unit oral doses of a compound or salt of this invention, and instructions for administering the compound or salt to reduce the uric acid concentration in blood of, or increasing uric acid excretion from, a mammalian subject.

Reducing uric acid as described herein can be used to treat or prevent a variety of conditions including gout (any or all of: asymptomatic gout, acute gouty arthritis, intercritical gout, and chronic tophaceous gout), hyperuricemia, elevated levels of uric acid that do not meet the levels customarily justifying a diagnosis of hyperuricemia, renal dysfunction, kidney stones, cardiovascular disease, risk for developing cardiovascular disease and other consequences of hyperuricemia, cognitive impairment, early-onset essential hypertension, and Plasmodium falciparum-induced inflammation.

This invention is based on the observation that compounds of this invention inhibited URAT1 in vitro, as shown in Example 3. Inhibition of URAT1 is an established in vitro model for lowering uric acid in vivo.

### DETAILED DESCRIPTION OF THE INVENTION

### Definitions

As used herein the term “alkyl” means a linear or branched-chain alkyl group. An alkyl group identified as having a certain number of carbon atoms means any alkyl group having the specified number of carbons. For example, an alkyl having three carbon atoms can be propyl or isopropyl; and an alkyl having four carbon atoms can be n-butyl, 1-methylpropyl, 2-methylpropyl or t-butyl.

As used herein the term “halo” refers to one or more of fluoro, chloro, bromo and iodo.

As used herein the term “perfluoro” as in perfluoromethyl or perfluoromethoxy, means that the group in question has fluorine atoms in place of all of the hydrogen atoms.

Certain chemical Compounds are referred to herein by their chemical name or by the two-letter code shown below. Compounds FF and FG are included within the scope of Formula I shown above.

FF 2-(3-(2,6-dimethylbenzyloxy)-4-methylphenyl)-2-oxoacetic acid
FG 2-(3-(2,6-dimethylbenzyloxy)-4-methoxyphenyl)-2-oxoacetic acid.

As used herein the transitional term “comprising” is open-ended. A claim utilizing this term can contain elements in addition to those recited in such claim.

As used in the claims the word “or” means “and/or” unless such reading does not make sense in context. So for example the phrase “reducing the uric acid concentration in blood of or increasing uric acid excretion from, a mammalian subject” is equivalent to “reducing the uric acid concentration in blood of and/or increasing uric acid excretion from, a mammalian subject.

The abbreviation S.D. means standard deviation. S.E. means standard error.

### Compounds of the Invention

In a more specific embodiment of the invention described above, R² is adjacent to the benzyloxy moiety and para to the oxoacetic acid/ester moiety. That is, the compound represented by Formula IA:

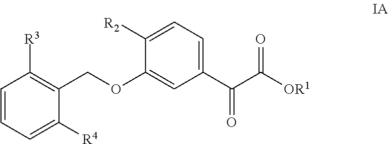

wherein R¹, R², R³, and R⁴ have the values set forth above, or a pharmaceutically acceptable salt of the compound. Further embodiments of this invention use a compound or salt of Formula I or IA in wherein R¹ is hydrogen; or wherein R² is methyl or methoxy; or wherein R³ is methyl and R⁴ is methyl; or wherein R¹ is hydrogen and R² is methyl or methoxy; or wherein R¹ is hydrogen, R³ is methyl, and R⁴ is methyl; or wherein R² is methyl or methoxy, R³ is methyl, and R⁴ is methyl; or wherein R¹ is hydrogen, R² is methyl or methoxy, R³ is methyl, and R⁴ is methyl. In more specific embodiments the compound is 2-(3-(2,6-dimethylbenzyloxy)-4-methylphenyl)-2-oxoacetic acid, or 2-(3-(2,6-dimethylbenzyloxy)-4-methoxyphenyl)-2-oxoacetic acid. In an embodiment of the compound of this invention, the compound or salt is in substantially (at least 98%) pure form.

### Reaction Schemes

The compound of formula I where R² is hydroxy, halo, alkoxy having from 1 to 3 carbon atoms, alkyl having from 1 to 3 carbon atoms, nitro, thio, alkylthio or cyano, R³ and R⁴ are independently hydrogen, methyl, ethyl, perfluoromethyl, methoxy, ethoxy, perfluoromethoxy, halo, hydroxy, nitro or amino, and R¹ is alkyl having from 1 to 3 carbon atoms, i.e. compounds of formula:

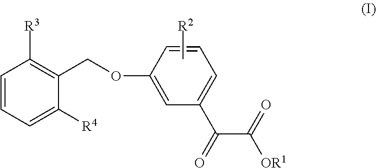

can be prepared via reaction scheme of Scheme 1. In the reaction scheme of Scheme 1, R¹, R², R³, and R⁴ are described as above. R⁵ is alkyl group having from 1 to 3 carbon atoms and Y is hydroxy or halo preferably iodo.

The compound of formula II can be converted to the compound of formula IV via reaction of step (a) by oxidation of methyl group with selenium dioxide (III) in the presence of pyridine. Generally the reaction is carried out at temperatures of from 25° C.-100° C. Any of the conditions conventional in such oxidation reactions can be utilized to carry out the reaction of step (a). The compound of formula IV is the compound of formula I where R¹ is H.

The compound of formula IV can be converted to the compound of formula VI by esterification of the compound of formula IV with methanol, ethanol or propanol. The reaction can be carried out either by using catalyst for example H₂SO₄, TsOH and the like or by alkylating the compound of formula IV with the compound of formula V using suitable base such as potassium carbonate, cesium carbonate and the like. Any of the conditions conventional in such esterification reactions can be utilized to carry out the reaction of step (b). The compound of formula VI is the compound of formula I where R¹ is alkyl having from 1 to 3 carbon atoms.

The products in all the steps can be isolated and purified by techniques such as extraction, evaporation, chromatography, and recrystallization.

If R², R³ and R⁴ is a hydroxy, thio or amino group, it is generally preferred to protect those groups by utilizing suitable protecting groups known to those skilled in the art. The suitable protecting group can be described in the Protective Groups in Organic Synthesis by T. Greene. The protecting group can be deprotected utilizing suitable deprotecting reagents such as those described in Protective Groups in Organic Synthesis by T. Greene.

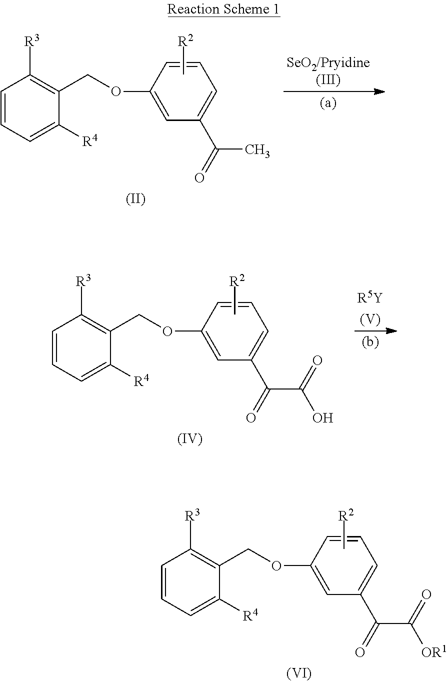

The compound of formula II where R² is halo, alkoxy having from 1 to 3 carbon atoms, alkyl having from 1 to 3 carbon atoms, nitro, or cyano and R³ and R⁴ are independently hydrogen, methyl, ethyl, perfluoromethyl, methoxy, ethoxy, perfluoromethoxy, halo, hydroxy, nitro or amino, i.e. compounds of formula:

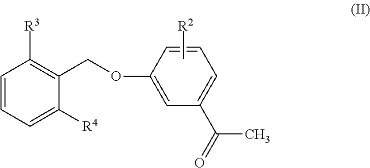

can be prepared via reaction scheme of Scheme 2. In the reaction scheme of Scheme 2, R², R³, and R⁴ are described as above. Z is a leaving group.

The compound of formula VII can be converted to the compound of formula II via reaction of step (c) using Mitsunobu condensation of VII with VIII using triphenylphosphine and diethyl azodicarboxylate or diisopropyl azodicarboxylate. The reaction can be carried out in a suitable solvent for example tetrahydrofuran. Any of the conditions conventionally used in Mitsunobu reactions can be utilized to carry out the reaction of step (c). The product can be isolated and purified by techniques such as extraction, evaporation, chromatography, and recrystallization.

The compound of formula II can also be prepared by etherifying or alkylating the compound of formula VII with the compound of formula IX via the reaction of step (d) by using suitable base such as potassium carbonate, cesium carbonate, sodium hydride, triethylamine, pyridine and the like. In the compound of formula IX, Z, include but are not limited to mesyloxy, tosyloxy, chloro, bromo, iodo, and the like. Any conventional conditions to alkylate a hydroxyl group with a leaving group can be utilized to carry out the reaction of step (d). The reaction of step (d) is preferred over step (c) if the compound of formula IX is readily available. The product can be isolated and purified by techniques such as extraction, evaporation, chromatography, and recrystallization.

If R², R³ or R⁴ is hydroxy or amino group, it is generally preferred to protect those groups by utilizing suitable protecting groups known to those skilled in the art. The suitable protecting groups are described in the Protective Groups in Organic Synthesis by T. Greene. The protecting group can be deprotected utilizing suitable deprotecting reagents such as those described in Protective Groups in Organic Synthesis by T. Greene.

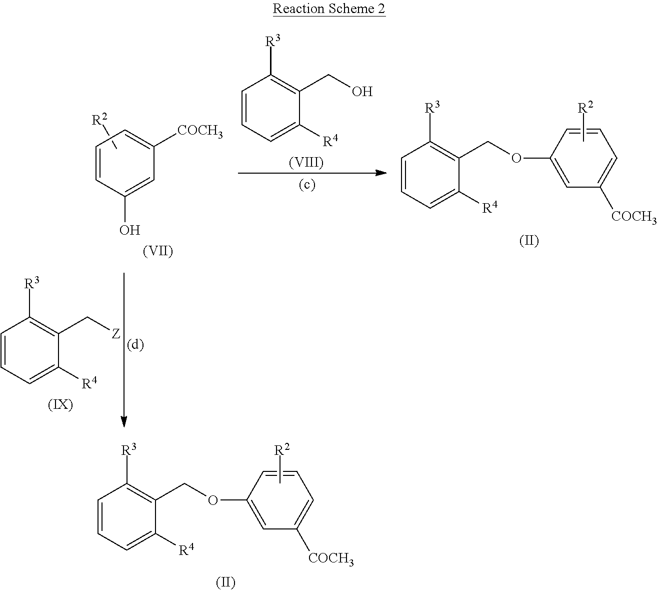

The compound of formula VII where R² is hydroxy, halo, alkoxy having from 1 to 3 carbon atoms, alkyl having from 1 to 3 carbon atoms, nitro, thio, alkylthio or cyano, i.e. compounds of formula:

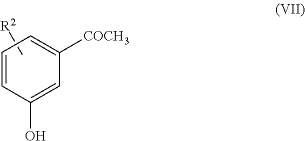

can be prepared via reaction scheme of Scheme 3. In the reaction of Scheme 3, R² is as above. The compound of formula VII can be synthesized according to the method of George M Rubottom et al., J. Org. Chem. 1983, 48, 1550-1552. The product can be isolated and purified by techniques such as extraction, evaporation, chromatography, and recrystallization.

If R² is a hydroxy or thio group, it is generally preferred to protect those groups by utilizing suitable protecting groups known to those skilled in the art. The suitable protecting groups are described in the Protective Groups in Organic Synthesis by T. Greene. The protecting group can be deprotected utilizing suitable deprotecting reagents such as those described in Protective Groups in Organic Synthesis by T. Greene.

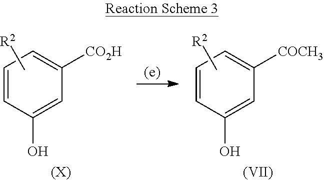

The compound of formula X where R² is halo or nitro, i.e. compounds of formula:

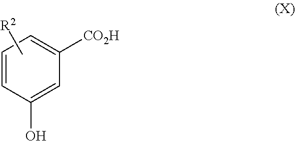

are either commercially available or can be prepared according to the methods described in the literature as follows:

### 1. 2-Br-3-OHC₆H₃CO₂H

### WO9628423.

### 2. 4-Br-3-OHC₆H₃CO₂H

### WO2001002388.

### 3. 3-Br-5-OHC₆H₃CO₂H

Journal of labeled Compounds and Radiopharmaceuticals, (1992), 31 (3), 175-82.

### 4. 2-Br-5-OHC₆H₃CO₂H

WO9405153 and U.S. Pat. No. 5,519,133.

### 5. 2-Cl-3-OHC₆H₃CO₂H

Proceedings of the Indiana Academy of Science, (1983), Volume date 1982, 92, 145-51.

### 6. 3-Cl-5-OHC₆H₃CO₂H

### WO2002000633 and WO2002044145.

### 7. 2-Cl-5-OHC₆H₃CO₂H

### WO9745400.

### 8. 2-I-3-OHC₆H₃CO₂H and 4-I-3-OHC₆H₃CO₂H

### WO9912928.

### 9. 5-I-3-OHC₆H₃CO₂H

J. Med. Chem. (1973), 16(6), 684-7.

The compound of formula X, where R² is alkoxy having from 1 to 3 carbon atoms and in which R² is adjacent to both the hydroxy and the carboxylic acid groups, i.e. compounds of formula:

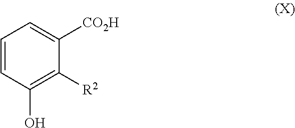

can be synthesized via the reaction of Scheme 4. In the reaction of Scheme 4, R⁵ is alkyl group having from 1 to 2 carbon atoms. P is a hydroxy protecting group.

The compound of formula XI can be converted to the compound of formula XII via reaction of step (f) by protecting phenol by suitable protecting group known to those skilled in the art. The suitable conditions for the protecting group are described in the Protective Groups in Organic Synthesis by T. Greene.

The compound of formula XII can be converted to the compound of formula XIII by oxidation of aldehyde to carboxylic acid. The reaction can be carried out by using suitable oxidizing reagents for example, pyridinium chlorochromate, potassium permanganate, sodium permanganate and the like. Any of the conditions suitable in such oxidation reactions can be utilized to carry out the reaction of step (g).

The compound of formula XIII can be converted to the compound of formula X via reaction of step (h) where R² is alkoxy having 1 carbon atom by deprotection of hydroxy protecting group. The suitable deprotecting conditions are described in the Protective Groups in Organic Synthesis by T Greene.

To make the compound of formula X in which R² is alkoxy having 2 or 3 carbon atoms, the compound of formula XIII can be converted to the compound of formula XIV by treating the compound of formula XIII with boron tribromide or boron trichloride using solvent for example dichloromethane for 4 to 48 hours at the temperature from

−72° C. to 0° C. Any of the conditions conventional in such reactions can be utilized to carry out the reaction of step (i).

The compound of formula XIV can be converted to the compound of formula XV by esterification of the compound of formula XIV with methanol or ethanol. The reaction can be carried out either by using catalysts for example H₂SO₄, TsOH and the like or by using dehydrating agent for example dicyclohexylcarbodiimide/DMAP and the like. Any of the conditions conventional in such esterification reactions can be utilized to carry out the reaction of step (j).

The compound of formula XV can be converted to the compound of formula XVI by etherifying or alkylating the compound of formula XV with alkyl halide having 2 to 3 carbon atoms in the presence of suitable base for example potassium carbonate, cesium carbonate, sodium hydride, pyridine and the like. The reaction can be carried out in conventional solvents, such as tetrahydrofuran, N,N-dimethylformamide, dichloromethane and the like. The reaction is generally carried out at temperatures from 0° C. to 40° C. Any of the conditions suitable in such alkylation reactions can be utilized to carry out the reaction of step (k).

The compound of formula XVI can be converted to the compound of formula X via reaction of step (l) where R² is alkoxy having 2 to 3 carbon atoms by deprotection of hydroxy protecting group. The suitable deprotecting conditions are described in the Protective Groups in Organic Synthesis by T Greene. The product can be isolated and purified by techniques such as extraction, evaporation, chromatography, and recrystallization.

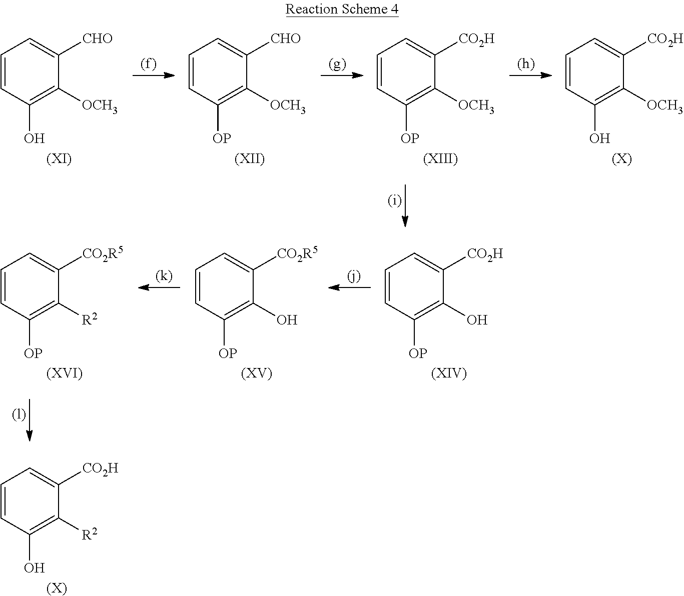

Alternatively, the compound of formula X, where R² is alkoxy having from 1 to 3 carbon atoms, i.e. compounds of formula:

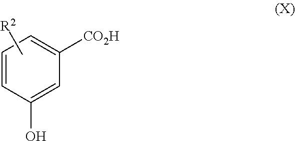

are either commercially available or can be prepared according to the methods described in the literature as follows:

### 1. 5-OMe-3-OHC₆H₃CO₂H

### J.O.C (2001), 66(23), 7883-88.

### 2. 2-OMe-5-OHC₆H₃CO₂H

U.S. Pat. No. 6,194,406 (Page 96), and Journal of the American Chemical Society (1985), 107(8), 2571-3.

### 3. 3-OEt-5-OHC₆H₃CO₂H

### Taiwan Kexue (1996), 49(1), 51-56.

### 4. 4-OEt-3-OHC₆H₃CO₂H

### WO 9626176

### 5. 2-OPr-5-OHC₆H₃CO₂H and 2-OEt-5-OHC₆H₃CO₂H

Adapt synthesis from U.S. Pat. No. 6,194,406 (Page 96) by using propyl iodide and ethyl iodide.

### 6. 4-OPr-3-OHC₆H₃CO₂H

Adapt synthesis from WO 9626176

### 7. 4-OEt-3-OHC₆H₃CO₂H

### Biomedical Mass Spectrometry (1985), 12(4), 163-9.

### 8. 3-OPr-5-OHC₆H₃CO₂H

Adapt synthesis from Taiwan Kexue (1996), 49(1), 51-56 by using propyl halide.

The compound of formula X, where R² is alkyl having from 1 to 3 carbon atoms, i.e. compounds of formula:

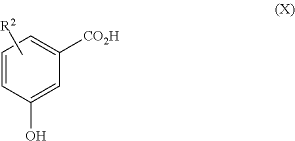

are either commercially available or can be prepared according to the methods described in the literature as follows:

### 1. 5-Me-3-OHC₆H₃CO₂H and 2-Me-5-OHC₆H₃CO₂H

### WO9619437,

### J.O.C. 2001, 66, 7883-88.

### 2. 2-Et-3-OHC₆H₃CO₂H

### JP10087489, WO9628423.

### 3. 4-Et-3-OHC₆H₃CO₂H

### J.O.C. 2001, 66, 7883-88.

### WO9504046.

### 4. 2-Et-5-OHC₆H₃CO₂H

### J.A.C.S. (1974), 96(7), 2121-9.

5. 2-nPr-3-OHC₆H₃CO₂H

### WO 9509843, WO 9628423.

6. 4-nPr-3-OHC₆H₃CO₂H

### WO9504046.

7. 2-nPr-5-OHC₆H₃CO₂H
Synthesis can be adapted from J.A.C.S (1974), 96(7), 2121-9 by using ethyl alpha formylvalerate.
8. 3-Et-5-OHC₆H₃CO₂H and 3-nPr-5-OHC₆H₃CO₂H
Adapt synthesis from J.O.C. 2001, 66, 7883-88 by using 2-Ethylacrolein and 2-Propylacrolein.

The compound of formula X, where R² is cyano, i.e. compounds of formula:

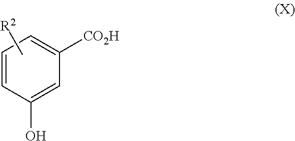

can be synthesized via the reaction of Scheme 5. In the reaction of scheme 5, R⁶ is alkyl group having from 1 to 2 carbon atoms. P₁ is hydroxy protecting group.

The compound of formula XVII can be converted to the compound of formula XVIII via reaction of step (m) by protecting phenol utilizing suitable protecting group known to those skilled in the art. The suitable conditions for the protecting group are described in the Protective Groups in Organic Synthesis by T. Greene.

The compound of formula XVIII can be reduced to the compound of formula XIX via reaction of step (n) by utilizing metals for example Zn, Sn, or Fe and acid, or by catalytic hydrogenation. Any conventional conditions in such reductions can be utilized to carry out the reaction of step (n).

The compound of formula XIX can be converted to the compound of formula XX via reaction of step (o) by various methods known to those skilled in the art for example, diazotization of amine using aqueous sulfuric acid at higher temperatures and then by adding aqueous sodium nitrite at 0-5° C. The intermediate aryl diazonium salt can be converted to the compound of formula XX either via Sandmeyer reaction using Cu(I) cyanide in DMF at high temperature or diazonium salt is further heated with aqueous sulfuric acid at 100-110° C. to give the hydroxy which can be converted to triflate. Palladium catalyzed displacement of the triflate with cyanide for example zinc cyanide in DMF can be utilized to give compound of formula XX. Any conventional conditions in such conversion of amine to nitrile can be utilized to carry out the reaction of step (o).

The compound of formula XX can be converted to the compound of formula XXI via reaction of step (p) by deprotection of hydroxy protecting group. The suitable deprotecting conditions are described in the Protective Groups in Organic Synthesis by T Greene.

The compound of formula XXI can be converted to the compound of X via reaction of step (q) by ester hydrolysis. Any conventional method of ester hydrolysis will produce the compound of formula X.

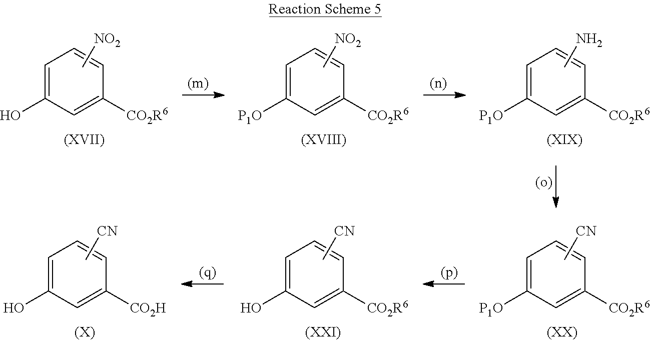

The compound of formula II, where R² is hydroxy, R³ and R⁴ are independently hydrogen, methyl, ethyl, perfluoromethyl, methoxy, ethoxy, perfluoromethoxy, halo, hydroxy, nitro or amino, i.e. compounds of formula:

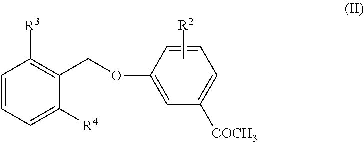

can be synthesized via the reaction of Scheme 6. In the reaction of scheme 6, R³ and R⁴ are described as above. P₁ and P₂ are hydroxy protecting groups.

The compound of formula XXII can be converted to the compound of formula XXIII via reaction of step (r) by protecting hydroxy group utilizing suitable protecting group known to those skilled in the art. The suitable conditions for the protecting group are described in the Protective Groups in Organic Synthesis by T. Greene.

The compound of formula XXIII can be converted to the compound of formula XXIV via reaction of step (s) by diazotization of amine using aqueous sulfuric acid at higher temperatures in the same manner as described hereinbefore in connection with the reaction of step (n). The compound of formula XXIV can be converted to the compound of formula XXV via reaction of step (t) by addition of aqueous sodium nitrite at 0-5° C. The intermediate aryl diazonium salt can be further heated with aqueous sulfuric acid at 100-110° C. to give the compound of formula XXV.

In the compound of formula XXV, hydroxy group can be protected by suitable protecting group known to those skilled in the art to give the compound of formula XXVI via reaction of step (u). The suitable conditions for the protecting group are described in the Protective Groups in Organic Synthesis by T. Greene.

The compound of formula XXVI can be converted to the compound of formula XXVII via reaction of step (v) by deprotection of selective hydroxy protecting group. The suitable deprotecting conditions are described in the Protective Groups in Organic Synthesis by T Greene.

The compound of formula XXVII can be converted to the compound of formula XXVIII via reaction of step (w) in the same manner as described hereinbefore in connection with the reaction of step (c).

The compound of formula XXVIII can be converted to the compound of formula II via reaction of step (x) by deprotection of hydroxy protecting group. The suitable deprotecting conditions are described in the Protective Groups in Organic Synthesis by T Greene. The product of each step can be isolated and purified by techniques such as extraction, evaporation, chromatography, and recrystallization.

If R³ and R⁴ are substituted by hydroxy or amino groups, it is generally preferred to protect those groups by utilizing suitable protecting groups known to those skilled in the art. The suitable protecting groups are described in the Protective Groups in Organic Synthesis by T. Greene. The protecting group can be deprotected utilizing suitable deprotecting reagents such as those described in Protective Groups in Organic Synthesis by T. Greene.

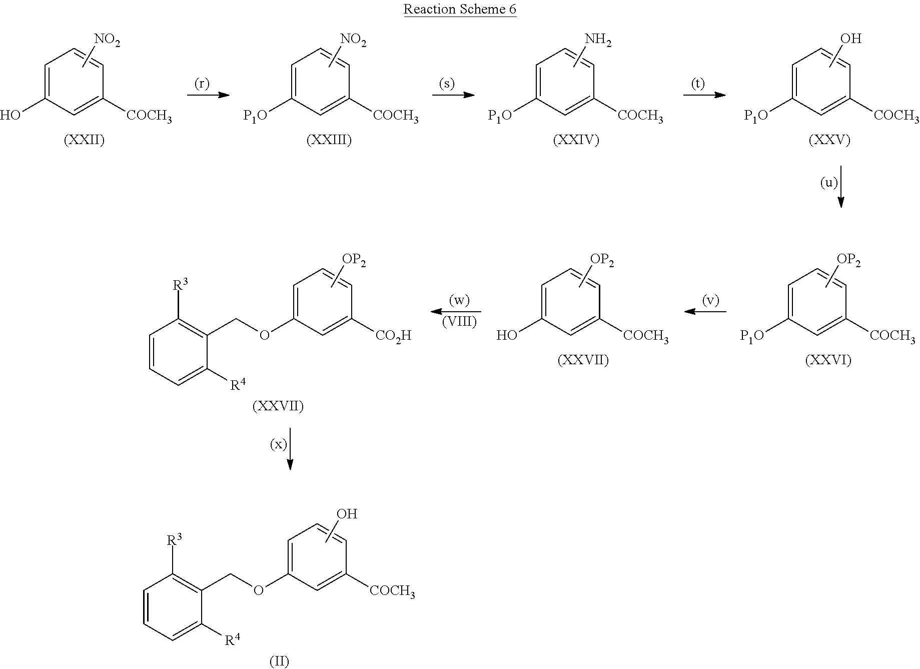

The compound of formula II, where R² is thio or alkylthio, i.e. compounds of formula:

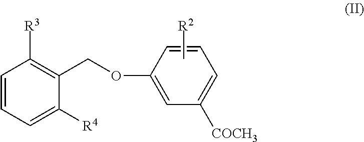

can be synthesized via the reaction of Scheme 7. In the reaction of Scheme 7, R³ and R⁴ are described as above. P₁ is hydroxy protecting group. R⁷ is alkyl group having from 1 to 3 carbon atoms.

The compound of formula XXIV can be converted to the compound of formula XXIX via reaction of step (y) by diazotization of amine using aqueous sulfuric acid at higher temperatures and then by adding aqueous sodium nitrite solution at 0-5° C. The intermediate aryl diazonium salt can be converted to the compound of formula XXIX by various methods known to those skilled in the art for example, utilizing Sandmeyer-type reaction of aryl diazonium salt with copper(I) salts such as copper(I) chloride, copper (I) bromide or using catalytic amount of copper salt with KI, KBr and the like. Any of the conditions conventional in such reactions can be utilized to carry the reaction of step (y).

The compound of formula XXIX can be converted to the compound of formula XXXI via reaction of step (z) by coupling the compound of formula XXIX with the compound of formula XXX utilizing palladium catalyst with ligands such as 1,1′-Bis(diphenylphosphino)ferrocene, (R,S)-1-[(1R,1S)-2-(Dicyclohexylphosphino) ferrocenyl]ethyldicyclohexylphosphine, or 1,2-Bis(diphenylphosphino)ethane. Any of the conditions conventional in such reactions can be utilized to carry the reaction of step (z).

The compound of formula XXXI can be converted to the compound of formula XXXIV via reaction of step (d′) where R² is alkylthio by deprotection of hydroxy protecting group. The suitable deprotecting conditions are described in the Protective Groups in Organic Synthesis by T Greene.

The compound of formula XXXIV can be converted to the compound of formula XXXV where R² is alkylthio via reaction of step (e′) in the same manner as described hereinbefore in connection with the reaction of step (c). The compound of formula XXXV is the compound of formula II where R² is alkylthio and R⁷ is alkyl group having from 1 to 3 carbon atoms.

The compound of formula XXIV can be converted to the compound of formula XXXII via reaction of step (a′) in the same manner as described hereinbefore with the connection with the reaction of step (t).

The compound of formula XXXII can be converted to the compound of formula XXXIII via reaction of step (b′) by reaction of the compound of formula XXXII with thioacetic acid in the presence TPP and diisopropylamine. Any of the conditions conventional in such reactions can be utilized to carry the reaction of step (b′).

The compound of formula XXXIII can be converted to the compound of formula XXXIV where R² is thio via reaction of step (c′) by deprotection of hydroxy protecting group. The suitable deprotecting conditions are described in the Protective Groups in Organic Synthesis by T Greene.

The compound of formula XXXIV can be converted to the compound of formula XXXV where R² is thio via reaction of step (e′) in the same manner as described hereinbefore in connection with the reaction of step (c).

The compound of formula XXXV can be converted to the compound of formula II where R² is thio via reaction of step (f′) by hydrolysis of compound of formula XXXV with sodium or potassium hydroxide. Any of the conditions conventional in such hydrolysis reactions can be utilized to carry the reaction of step (f′). The product of each step can be isolated and purified by techniques such as extraction, evaporation, chromatography, and recrystallization.

If R³ and R⁴ are hydroxy or amino groups, it is generally preferred to protect those groups by utilizing suitable protecting groups known to those skilled in the art. The suitable protecting groups are described in the Protective Groups in Organic Synthesis by T. Greene. The protecting group can be deprotected utilizing suitable deprotecting reagents such as those described in Protective Groups in Organic Synthesis by T. Greene.

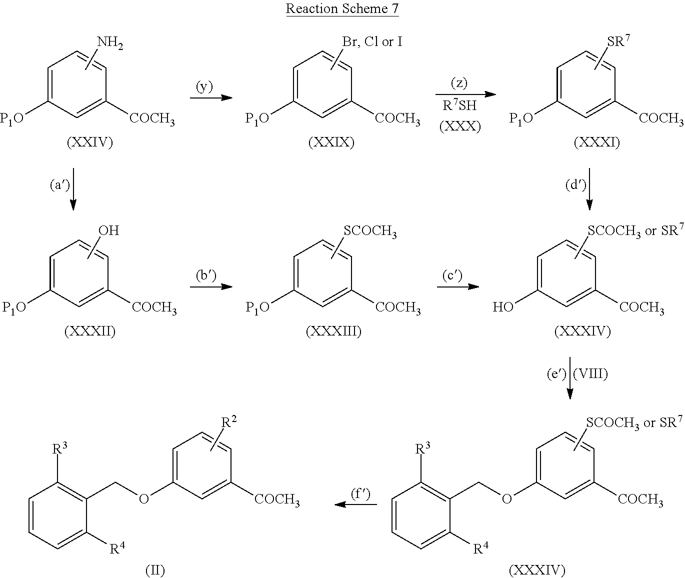

The compound of formula VIII, where R³ and R⁴ are independently hydrogen, methyl, ethyl, perfluoromethyl, methoxy, ethoxy, perfluoromethoxy, halo, hydroxy, nitro or amino, i.e. compounds of formula:

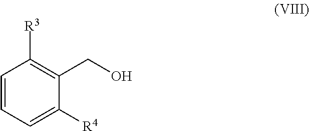

and the compound of formula IX, where R³ and R⁴ are described as above, i.e. compounds of formula:

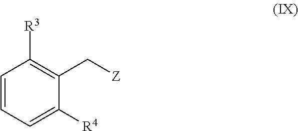

can be prepared via reaction scheme of Scheme 8. In the reaction scheme of Scheme 8, R³ and R⁴ are described as above. Z is a leaving group.

The compound of formula XXXVI can be reduced to the compound of formula VIII via reaction of step (g′). The reaction can be carried out utilizing a conventional reducing agent for example alkali metal hydride such as lithium aluminum hydride, sodium borohydride or borane dimethyl sulfide complex and the like. The reaction can be carried out in a suitable solvent, such as tetrahydrofuran, ether, and the like. Any of the conditions conventional in such reduction reactions can be utilized to carry out the reaction of step (g′).

The compound of formula VIII can be converted to the compound of formula IX by displacing hydroxy group with a leaving group, such as tosyloxy, mesyloxy or halogen group preferred halogen being bromo or chloro. Appropriate halogenating reagents include but are not limited to thionyl chloride, oxalyl chloride, bromine, phosphorous tribromide, carbon tetrabromide and the like. Any conditions conventional in such reactions can be utilized to carry out the reaction of step (h′). The product of the each step can be isolated and purified by techniques such as extraction, evaporation, chromatography, and recrystallization.

If R³ and R⁴ are phenyl substituted by hydroxy or amino groups, it is generally preferred to protect those groups by utilizing suitable protecting groups known to those skilled in the art. The suitable protecting group can be described in the Protective Groups in Organic Synthesis by T. Greene. The protecting group can be deprotected utilizing suitable deprotecting reagents such as those described in Protective Groups in Organic Synthesis by T. Greene.

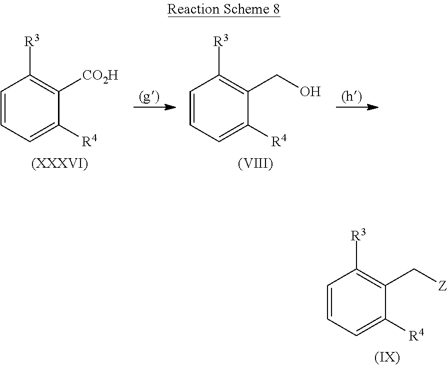

### Uses and Methods of Treatment

This invention provides a method for reducing uric acid levels in a mammalian subject or increasing uric acid excretion from a mammalian subject. The level of uric acid in a mammal can be determined using any conventional measure. Typically the level of uric acid in the blood is determined Uric acid can also be deposited or precipitated in tissues, resulting in depots (e.g. tophi) that can be affected by raising or lowering blood uric acid concentrations, and which conversely can contribute to circulating uric acid. The method of this invention for reducing uric acid can be used to treat or prevent a variety of conditions including gout, hyperuricemia, elevated levels of uric acid that do not meet the levels customarily justifying a diagnosis of hyperuricemia, kidney stones, renal dysfunction, cardiovascular disease, cardiovascular risk factor, and cognitive impairment. By lowering uric acid levels, administration of the compounds of this invention slows progression of kidney disease. An elevated uric acid level has been identified as a risk factor for cardiovascular disease. A significant correlation has been shown between elevated uric acid and cognitive impairment in older adults. (Schretlen, D. J. et al., “Serum Uric Acid and Cognitive Function in Community-Dwelling Older Adults”, Neuropsychology (January 2007) 21(1): 136-140). Accordingly, the method of this invention for reducing uric acid can be used to treat or prevent cognitive impairment, including cognitive impairment in elderly adults. It is well known that people with Lesch-Nyhan Syndrome have elevated levels of uric acid and suffer the numerous consequences of this hyperuricemia, including gout. Thus, this invention for reducing blood levels and increasing elimination of uric acid can be used to treat people with Lesch-Nyhan Syndrome.

The normal range of uric acid in blood is between 3.4 mg/dL and 7.0 mg/dL in men, between 2.4 mg/dL and 6.0 mg/dL in premenopausal women, and from 2.5 mg/dL to 5.5 mg/dL in children. Urate crystal formation/precipitation typically occurs in men at levels of 6.6 mg/dL or higher and in women at levels of 6.0 mg/dL or higher. This illustrates that levels of uric acid that are within the so-called normal range can have undesirable health consequences, even producing gout. Also, what may be in the normal range for the population as a whole may be elevated for the individual. Cardiovascular and other consequences of elevated uric acid can occur with blood levels well within these “normal” ranges. Therefore, a diagnosis of hyperuricemia is not necessarily a prerequisite for the beneficial effects of the compounds of the invention.

This invention includes the treatment of hyperuricemia associated with gout, hypertension, vascular inflammation, heart failure, arterio-venous disorders, myocardial infarct, stroke, pre-eclampsia, eclampsia, sleep apnea, renal dysfunction (including renal failure, end stage renal disease [ESRD]), organ transplant, diuretics, thiazides, cyclosporine, aspirin, vitamin C, nicotinic acid, levodopa (L-DOPA), cytotosic drugs, and certain antibacterial agents (such as pyrozinamide), cirrhosis, thyroid dysfunction, parathyroid dysfunction, lung cancer, anemia, leukemia, lymphoma, multiple myeloma, tumor-lysis syndrome, thyroid or parathyroid dysfunction, Lesch-Nyhan Syndrome, smoking, alcohol consumption, and psoriasis. This invention includes the treatment of hyperuricemia that can lead to gout, formation of urate crystals, renal dysfunction, graft or organ failure following transplant, endothelial disorders (such as inflammation), chronic heart failure, arterio-venous disorders, pre-eclampsia, eclampsia, hypertension, and cognitive impairment. In embodiments of the method of this invention for treating gout, tissue deposits of uric acid, including but not limited to tophi, are reduced, and the incidence and severity of gout flares are also reduced.

The compounds of this invention can be administered by any conventional route of systemic administration. Preferably they are administered orally. Accordingly, it is preferred for the medicament to be formulated for oral administration. Other routes of administration that can be used in accordance with this invention include rectally, parenterally, by injection (e.g. intravenous, subcutaneous, intramuscular or intraperitioneal injection), or nasally.

Further embodiments of each of the uses and methods of treatment of this invention comprise administering any one of the embodiments of the compounds described above. In the interest of avoiding unnecessary redundancy, each such compound and group of compounds is not being repeated, but they are incorporated into this description of uses and methods of treatment as if they were repeated.

Both human and non-human mammalian subjects can be treated in accordance with the treatment method of this invention. The optimal dose of a particular compound of the invention for a particular subject can be determined in the clinical setting by a skilled clinician. In the case of oral administration the compound of this invention is generally administered to adults in a daily dose of from 1 mg to 2500 mg, more preferably from 1 mg to 1200 mg. In other embodiments of this invention the compound is administered in a dose of from 400 mg to 1000 mg, from 600 mg to 800 mg, from 600 mg to 1000 mg, or from 100 to 300 mg, administered once or twice per day. The average body weight of a typical adult is 60 to 70 kilograms, so that appropriate dose ranges expressed as mg/kg are approximately from 0.015 to 42 mg/kg, from 0.015 to 20 mg/kg, from 6.6 to 13 mg/kg, from 10 to 13 mg/kg, from 10 to 16 mg/kg, or from 1.67 to 4.3 mg/kg, administered once or twice per day. When treating children the optimal dose is determined by the patient's physician. In the case of oral administration to a mouse the compound of this invention is generally administered in a daily dose from 1 to 300 mg of the compound per kilogram of body weight.

The compound of this invention can be administered in combination with other uric acid lowering drugs. In such cases the dose of the compound of this invention is as described above. Any conventional or investigational uric acid lowering drug can be utilized in combination with the compound of this invention. Examples of such drugs include xanthine oxidase inhibitors such as allopurinol (from 100 mg/day to 1000 mg/day; more typically from 100 mg/day to 300 mg/day) febuxostat (from 40 mg/day to 120 mg/day; more specifically from 60 mg/day to 80 mg/day) and oxypurinol; Puricase/PEG-uricase (from 4 mg to 12 mg every two weeks by infusion); uricosuric agents such as sulfinpyrazone (from 100 mg/day to 800 mg/day), probenecid (500 mg/day), losartan (from 25 mg/day to 200 mg/day, more typically from 50 mg/day to 100 mg/day), fenofibrate, JTT-552 (a URAT-1 inhibitor), benzbromarone (from 70 mg/day to 150 mg/day), and statins such as atorvastatin (LIPITOR®). The other uric acid lowering drug can be administered in its usual amount or in an amount that is less than the usual amount, whether by administering lower doses of such other drug or by less frequent dosing with such other drug.

The compounds of this invention can be administered together with other drugs used to decrease the pain associated with gouty attacks, for example nonsteroidal antiinflammatory drugs (NSAIDs), colchicine, corticosteroids, and other analgesics.

In the course of lowering uric acid levels in the blood it is expected that the compounds of this invention will increase the levels of uric acid in the urine. To increase the pH of the urine and thereby improve solubility of the uric acid, citrate or bicarbonate, for example, can be administered in conjunction with the compound of this invention.

An admixture of the compound or salt of this invention with one or more other uric acid lowering drugs, analgesics, and pH increasing agents, can be administered to the subject. Alternatively the compound or salt of this invention and the one or more other uric acid lowering drugs, analgesics, and pH increasing agents are not mixed together to form an admixture but are administered independently to the subject. When the active ingredients are not mixed together to form a single admixture or composition it is convenient to provide them in the form of a kit comprising one or more unit oral doses of a compound of this invention, one or more unit oral doses of one or more other uric acid lowering drugs, analgesics, and pH increasing agents, and instructions for administering the compound of this invention in combination with the other active ingredients. Preferably the components of the kit are packaged together, such as in a box or a blister pack.

### Pharmaceutical Compositions

This invention provides a pharmaceutical composition comprising a compound of this invention, and optionally a pharmaceutically acceptable carrier. Further embodiments of the pharmaceutical composition of this invention comprise any one of the embodiments of the compounds described above. In the interest of avoiding unnecessary redundancy, each such compound and group of compounds is not being repeated, but they are incorporated into this description of pharmaceutical compositions as if they were repeated.

Preferably the composition is adapted for oral administration, e.g. in the form of a tablet, coated tablet, dragee, hard or soft gelatin capsule, solution, emulsion or suspension. In general the oral composition will comprise from 1 mg to 2500 mg, more preferably from 1 mg to 1200 mg of the compound of this invention. In more specific embodiments of this invention the oral composition will comprise from 400 mg to 1000 mg, from 600 mg to 800 mg, from 600 mg to 1000 mg, or from 100 to 300 mg, of the compound of this invention. It is convenient for the subject to swallow one or two tablets, coated tablets, dragees, or gelatin capsules per day. However the composition can also be adapted for administration by any other conventional means of systemic administration including rectally, e.g. in the form of suppositories, parenterally, e.g. in the form of injection solutions, or nasally.

The active ingredients can be processed with pharmaceutically inert, inorganic or organic carriers for the production of pharmaceutical compositions. Lactose, corn starch or derivatives thereof, talc, stearic acid or its salts and the like can be used, for example, as such carriers for tablets, coated tablets, dragees and hard gelatin capsules. Suitable carriers for soft gelatin capsules are, for example, vegetable oils, waxes, fats, semi-solid and liquid polyols and the like. Depending on the nature of the active ingredient no carriers are, however, usually required in the case of soft gelatin capsules, other than the soft gelatin itself. Suitable carriers for the production of solutions and syrups are, for example, water, polyols, glycerol, vegetable oils and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, coating agents or antioxidants.

The invention will be better understood by reference to the following examples, which illustrate but do not limit the invention described herein.

### EXAMPLES

### Example 1

### Synthesis of 2-(3-(2,6-Dimethylbenzyloxy)-4-Methylphenyl)-2-Oxoacetic Acid

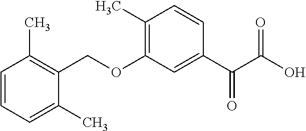 [SMILES: Cc1ccc(C(=O)C(=O)O)cc1OCc1c(C)cccc1C]

### 2-(3-(2,6-dimethylbenzyloxy)-4-methylphenyl)-2-oxoacetic acid

### Step A: Preparation of 1-(3-amino-4-methylphenyl)ethanone

To a stirred solution of 1-(4-methyl-3-nitrophenyl)ethanone (11.51 g, 55 mmol) in abs ethanol (200 ml) was added tin dichloride dihydrate (50 g, 220 mmol), and the reaction mixture was heated at 80° C. for 3 hours or until all the starting material is consumed. The reaction mixture was cooled, concentrated and pH of the crude mixture was adjusted to 5 by addition of 5N NaOH. The solids were filtered and washed with ethyl acetate (3×). The combined organic layer was washed with water, brine, dried over Na₂SO₄, filtered, concentrated, and purified by flash chromatography on a silica gel column (chloroform:methanol, 95:5) to give the title compound as solid.

¹H NMR (400 MHz, CDCl₃): 2.20 (s, 3H); 2.54 (s, 3H); 3.90 (b, 2H); 7.15 (d, 1H); 7.28 (m, 2H).

### Step B: Preparation of 1-(3-hydroxy-4-methylphenyl)ethanone

The solution of 1-(3-amino-4-methylphenyl)ethanone (Step A, 1.95 g, 13.1 mmol) in conc. H₂SO₄ (3.56 ml) and water (15 ml) was heated to a homogenous solution, then cooled to 0° C. and diluted with water (35 ml). To the reaction mixture was added drop wise a solution of NaNO₂ (0.924 g, 13.4 mmol) in water (2 ml) at 0° C. and then mixture was allowed to stir at the same temperature for 30 min to give diazonium intermediate.

The above diazonium reaction mixture was added drop wise to a heated solution of conc. H₂SO₄ (14 ml) and water (21 ml) and reaction mixture was further heated to 120° C. for another 20-25 min, cooled to room temperature, and then extracted with ethyl acetate (4×). The combined organic layer was washed with brine, dried over Na₂SO₄, filtered, concentrated, and purified by flash chromatography on a silica gel column (hexane:ethyl acetate, 2:1) to give the title compound as an oil.

¹H NMR (400 MHz, CDCl₃): 2.21 (s, 3H); 2.57 (s, 3H); 7.19-7.21 (d, 1H); 7.42-7.51 (m, 2H).

### Step C: Preparation of 1-(3-(2,6-dimethylbenzyloxy)-4-methylphenyl)ethanone

To a stirred solution of 1-(3-hydroxy-4-methylphenyl)ethanone (Step B, 0.841 g, 5.6 mmol), and K₂CO₃ (1.55 g, 11.2 mmol) in dry DMF (10 ml) was added 2,6-dimethylbenzyl chloride (0.8843 g, 5.7 mmol) under argon. The reaction mixture was stirred at ambient temperature for 16 hours, diluted with ethyl acetate, washed with water (2×), and brine. The organic layer was dried over Na₂SO₄, filtered, concentrated, and purified by flash chromatography on a silica gel column (hexane:ethyl acetate, 2:1) to give the title compound as off white solid.

¹H NMR (400 MHz, CDCl₃): 2.21 (s, 3H); 2.38 (s, 6H); 2.57 (s, 3H); 5.10 (s, 2H); 7.07 (m, 2H); 7.09 (m, 2H); 7.5 (d, 1H); 7.63 (s, 1H).

### Step D: Preparation of 2-(3-(2,6-dimethylbenzyloxy)-4-methylphenyl)-2-oxoacetic acid

To a stirred solution of 1-(3-(2,6-dimethylbenzyloxy)-4-methylphenyl)ethanone (Step C, 1.40 g, 5.22 mmol) in pyridine (25 ml) was added selenium dioxide (3.78 g, 34 mmol) and the reaction mixture was heated at 100° C. for 3 hours or until all the starting material is consumed. The reaction mixture is decanted from selenium metal and concentrated to give orange semisolid. The crude residue was taken in chloroform and washed with 1M HCl to bring aqueous pH to 4. The organic layer was dried over Na₂SO₄, filtered, concentrated, and purified by flash chromatography on a silica gel column (chloroform:methanol, 92.5:7.5 spiked with acetic acid) to give the title compound as light yellow solid.

¹H NMR (400 MHz, d₆-DMSO): 2.21 (s, 3H); 2.38 (s, 6H); 5.10 (s, 2H); 7.20 (m, 2H); 7.31 (m, 2H); 7.5 (d, 1H); 7.63 (s, 1H).

### Example 2

### Synthesis of 2-(3-(2,6-Dimethylbenzyloxy)-4-Methoxyphenyl)-2-Oxoacetic Acid

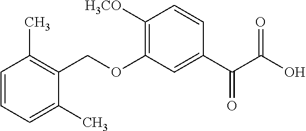 [SMILES: COc1ccc(C(=O)C(=O)O)cc1OCc1c(C)cccc1C]

### 2-(3-(2,6-dimethylbenzyloxy)-4-methoxyphenyl)-2-oxoacetic acid

### Step A: Preparation of 1-(3-(2,6-dimethylbenzyloxy)-4-methoxyphenyl)ethanone

To a stirred solution of 1-(3-hydroxy-4-methoxyphenyl)ethanone (5 g, 30 mmol), and K₂CO₃ (8.32 g, 60.2 mmol) in dry DMF (20 ml) was added 2,6-dimethylbenzyl chloride (4.89 g, 31.6 mmol) under argon. The reaction mixture was stirred at ambient temperature for 16 hours, diluted with ethyl acetate, washed with water (2×), and brine. The organic layer was dried over Na₂SO₄, filtered, concentrated, and purified by flash chromatography on a silica gel column (hexane:ethyl acetate, 2:1) to give the title compound as off white solid.

¹H NMR (400 MHz, CDCl₃): 2.38 (s, 6H); 2.57 (s, 3H); 3.83 (s, 3H); 5.09 (s, 2H); 7.04 (d, 2H); 7.15-7.17 (m, 2H); 7.62 (d, 1H); 7.63 (s, 1H).

### Step B: Preparation of 2-(3-(2,6-dimethylbenzyloxy)-4-methoxyphenyl)-2-oxoacetic acid

To a stirred solution of 1-(3-(2,6-dimethylbenzyloxy)-4-methoxyphenyl)ethanone (Step A, 2.72 g, 9.5 mmol) in pyridine (45 ml) was added selenium dioxide (6.92 g, 62.4 mmol) and the reaction mixture was heated at 100° C. for 3 hours or until all the starting material is consumed. The reaction mixture is decanted from selenium metal and concentrated to give orange semisolid. The crude residue was taken in chloroform and washed with 1M HCl to bring aqueous pH to 4. The organic layer was dried over Na₂SO₄, filtered, concentrated, and purified by flash chromatography on a silica gel column (chloroform:methanol, 9:1 spiked with acetic acid) to give the title compound as light yellow solid.

¹H NMR (400 MHz, d₆-DMSO): 2.31 (s, 6H); 3.83 (s, 3H); 5.09 (s, 2H); 7.20 (m, 2H); 7.31 (m, 2H); 7.5 (d, 1H); 7.63 (s, 1H).

### Example 3

### URAT1 Inhibition Assay

URAT1 (Uric Acid Transporter 1) is expressed on the apical membrane in renal tubules. It mediates the re-uptake of uric acid from the urine into the blood. Inhibition of URAT1 leads to increased excretion of uric acid in the urine, and is therefore a potential mode of action for drugs that lower serum uric acid concentrations. Probenecid and Benzbromarone, for example, have been used clinically for treatment of gout and hyperuricemia, and they both act on URAT1 to reduce uric acid reuptake. However, benzbromarone was withdrawn from the market due to liver toxicity via mechanisms independent of URAT1, and probenecid acts on numerous transporter proteins, resulting in interactions with a variety of other drugs.

An in vitro URAT1 assay is useful for identifying compounds with potential activity in lowering serum uric acid. A suitable assay involves transfection of cells (e.g. human embryonic kidney cells; “HEK”) with a vector encoding human URAT1, followed by determination of the ability of transfected cells to take up radiolabeled uric acid. The activity of compounds as URAT1 inhibitors is evaluated by their ability to block uric acid uptake by transfected cells.

### Test Compounds and Chemicals:

[8-¹⁴C] Urate (50-60 mCi/mmol; American Radio Chemicals, Cat. No. ARC0513).

Subcloning of hURAT1 into the Expression Vector:

Plasmid vector pCMV6-XL5 containing hURAT1 cDNA (Cat. No. SC125624) and the expression vector pCMV6-Neo (Cat. No. pCMVNEO) were obtained from OriGene Technologies, Inc. The full-length hURAT1 cDNA was obtained from the vector pCMV6-XL5 and subcloned into the expression vector pCMV6-Neo to create the hURAT1 expression plasmid pCMV6-hURAT1. The sequences were verified by automatic DNA sequencing.

Cell Culture, Transfection of URAT1 Expressing Plasmids and the Establishment of Stably Expressing HEK Cells for hURAT1:

Human embryonic kidney 293 (HEK) cells (ATTCC, Cat No. CRL-1573) were cultured in EMEM supplemented with 10% FBS and 2 mM L-glutamine and incubated at 37° C. and 5% CO₂. For transfection experiments, cells were plated on 60 mm dishes in 1 ml media per dish. After an 18-24 hour incubation, cells were transfected with plasmid pCMV6-hURAT1 or the expression vector pCMV6-Neo, using the Lipofectin trasfection agent following the manufacturer's instructions (Invitrogen, Cat. No. 18292). After transfection cells were grown in EMEM media for 72 hours and then by adding 1 mg/ml Geneticin (GIBCO, Cat. No 10131) stable transfectants were selected. Stable transfectants expressing hURAT1 (herein after referred as hURAT1-HEK cells) or cells having only the expression vector pCMV6-Neo (herein after referred as mock-HEK cells) were verified using reverse transcription polymerase chain reaction (RT-PCR) methods.

### [8-¹⁴C] Urate Uptake Assay:

hURAT1-HEK cells and mock-HEK cells were plated in poly-D-Lysine Cell culture 24 well plates (Becton Dickinson, Cat. No. 354414) at a concentration of 3×10⁵ in EMEM medium and incubated overnight. Reaction solutions containing the [8-¹⁴C] urate (55 mCi/mmol) at a final concentration of 50 μM were prepared with or without test compounds in Hanks' balanced salt solution (HBSS) containing 125 mM sodium gluconate, 4.8 mM potassium gluconate, 1.3 mM calcium, 5.6 mM glucose, 1.2 mM magnesium sulfate, 1.2 mM KH₂PO₄ and 25 mM HEPES (pH7.4). Before the uptake assay started, the culture medium was removed and the cells were incubated for 5 min in 0.6 ml of HBSS. After that HBSS was removed, the prepared reaction solutions were added into each well and incubated for 5 min at room temperature. Then the reaction solution was removed, cells were washed twice with 0.6 ml of cold HBSS and lysed with 0.2 ml of 0.1 M NaOH for 20 min. The cell lysates were transferred into the scintillation vials containing 1 ml of scintillation fluid (Opti Phase SuperMIX, PerkinElmer, Cat No. 1200-439) and the radioactivity was counted in the Microbeta counter (1450, Wallac Jet, PerkinElmer). Test compounds were dissolved in DMSO and the same concentration of DMSO was added into the wells of mock-HEK cells and the hURAT1-HEK cells that didn't contain test compounds. For each test compound, the uptake assay was performed 2 times and carried out in triplicate. Urate uptake of the cells for each test condition was presented as the average percent inhibition in comparison to the DMSO control. The radioactivity values obtained for the wells that contained DMSO were taken as 100% uptake of the cells. The observed concentration—percent inhibition data were fitted to a sigmoidal concentration-effect model, where:

% Inhibition=(100*ConĉSlope)/(IC50̂Slope+ConĉSlope)

IC₅₀ and slope estimates with their 95% confidence limits were determined by a non-linear, least-squares regression analysis using the Data Analysis Toolbox™ (MDL Information Systems, San Leandro, Calif., USA).

For assessment of activity of compounds as URAT1 inhibitors, the percent inhibition of uric acid uptake was typically assessed at a drug concentration of 10 micromolar (Table 1). Additional drug concentrations of compound FF was tested for determination of IC-50 values (Table 2).

TABLE 1
Inhibitory Effects of Various Compounds (at 10 μM) on Uric Acid
Uptake in Human URAT1-Expressing HEK293 cells

| | % Inhibition | S.D. | S.E. |
|---|---|---|---|
| Cpd. FF | 87.42 | 1.7 | 0.98 |
| Cpd. FG | 67.07 | 2.15 | 1.24 |

TABLE 2

| | IC50 (μM) |
|---|---|
| Cpd. FF | 1.28 |

1. A compound represented by Formula I 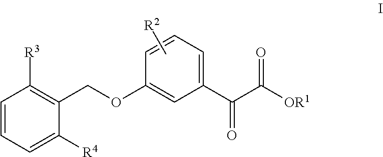
wherein
R¹ is hydrogen or alkyl having from 1 to 3 carbon atoms;
R² is alkyl having from 1 to 3 carbon atoms, alkoxy having from 1 to 3 carbon atoms, hydroxy, nitro, halo, thio, alkylthio, or cyano;
R³ and R⁴ are each independently hydrogen, methyl, ethyl, perfluoromethyl, methoxy, ethoxy, perfluoromethoxy, halo, hydroxy, nitro, or amino;
or a pharmaceutically acceptable salt of the compound.

2. The compound of claim 1 represented by Formula IA 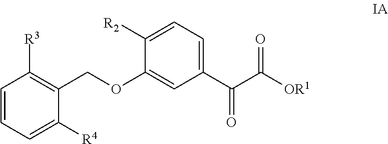
wherein R¹, R², R³, and R⁴ have the values set forth in claim 1,
or a pharmaceutically acceptable salt of the compound.

3. The compound or salt of claim 2, wherein R¹ is hydrogen.

4. The compound or salt of claim 3, wherein R³ is methyl and R⁴ is methyl.

5. The compound or salt of claim 4, wherein R² is methyl or methoxy.

6. The compound or salt of claim 5, wherein the compound is 2-(3-(2,6-dimethylbenzyloxy)-4-methylphenyl)-2-oxoacetic acid.

7. The compound or salt of claim 5, wherein the compound is 2-(3-(2,6-dimethylbenzyloxy)-4-methoxyphenyl)-2-oxoacetic acid.

8. (canceled)

9. The compound or salt of claim 2, wherein R² is methyl or methoxy.

10. (canceled)

11. The compound or salt of claim 2, wherein R³ is methyl and R⁴ is methyl.

12. A method of reducing the uric acid concentration in blood of, or increasing uric acid excretion from, a mammalian subject, comprising administering to the subject a compound or salt of claim 1, in an amount effective to reduce the uric acid concentration in blood of, or increase uric acid excretion from, the subject.

13. A method according to claim 12, for treating or preventing a condition selected from the group consisting of gout, hyperuricemia, elevated levels of uric acid that do not meet the levels customarily justifying a diagnosis of hyperuricemia, renal dysfunction, kidney stones, cardiovascular disease, risk for developing cardiovascular disease, tumor-lysis syndrome, cognitive impairment, early-onset essential hypertension, and Plasmodium falciparum-induced inflammation.

14. The method of claim 12, wherein the subject is a human.

15. The method of claim 12, further comprising administering to the subject one or more other uric acid lowering drugs in a combined amount effective to reduce the uric acid concentration in blood of, or increase uric acid excretion from, the subject.

16. The method of claim 15, wherein the other uric acid lowering drug is selected from the group consisting of a xanthine oxidase inhibitor, a uricosuric agent, a urate transporter-1 inhibitor, a uricase, and a statin.

17. The method of claim 12, wherein the compound is formulated for oral administration.

18-27. (canceled)

28. A pharmaceutical composition for use in reducing the uric acid concentration in blood of, or increasing uric acid excretion from, a mammalian subject, comprising a pharmaceutically acceptable carrier and a compound or salt of claim 1, in an amount effective to reduce the uric acid concentration in blood of, or increase uric acid excretion from, the subject.

29. The pharmaceutical composition of claim 28, for use in treating or preventing a condition selected from the group consisting of gout, hyperuricemia, elevated levels of uric acid that do not meet the levels customarily justifying a diagnosis of hyperuricemia, renal dysfunction, kidney stones, cardiovascular disease, risk for developing cardiovascular disease, tumor-lysis syndrome, cognitive impairment, early-onset essential hypertension, and Plasmodium falciparum-induced inflammation.

30-32. (canceled)

33. The pharmaceutical composition of claim 28, formulated for oral administration.
